(19) United States
(12) Patent Application Publication
(10) Pub. No.: US 2014/0086861 A1
(43) Pub. Date: Mar. 27, 2014

(54) HYDROPHILIC TREATED PIGMENTS DISPERSIBLE IN A COSMETIC COMPOSITION

(71) Applicants: Sophie Goldlum, Pierrefitte sur Seine (FR); Antoine Lecuivre, Saint Ouen L'Aumone (FR); Gaelle Frere, Saint Germain en Laye (FR); Virginie Boulier, Condecourt (FR); Emilie Lasjaunias, Magny en Vexin (FR); Christelle Mouix, Cergy (FR); Christine Geneve, Triel Sur Seine (FR)

(72) Inventors: Sophie Goldlum, Pierrefitte sur Seine (FR); Antoine Lecuivre, Saint Ouen L'Aumone (FR); Gaelle Frere, Saint Germain en Laye (FR); Virginie Boulier, Condecourt (FR); Emilie Lasjaunias, Magny en Vexin (FR); Christelle Mouix, Cergy (FR); Christine Geneve, Triel Sur Seine (FR)

(73) Assignee: SENSIENT COSMETIC TECHNOLOGIES, Saint Ouen L'Aumône (FR)

(21) Appl. No.: 14/003,754

(22) PCT Filed: Mar. 8, 2012
(86) PCT No.: PCT/EP2012/054045

§ 371 Date: Dec. 10, 2013

(30) Foreign Application Priority Data
Mar. 9, 2011 (FR) 1151922

(51) Int. Cl. A61K 8/55; A61Q 1/10; A61Q 5/06; A61Q 1/08

(52) U.S. Cl. CPC A61K 8/55; A61Q 1/08; A61Q 1/10; A61Q 5/065
USPC 424/63; 424/70.7; 424/70.6

(57) ABSTRACT

The invention relates to a pigment composition comprising:
a pigment,
an additive of the following formula (I):

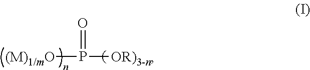

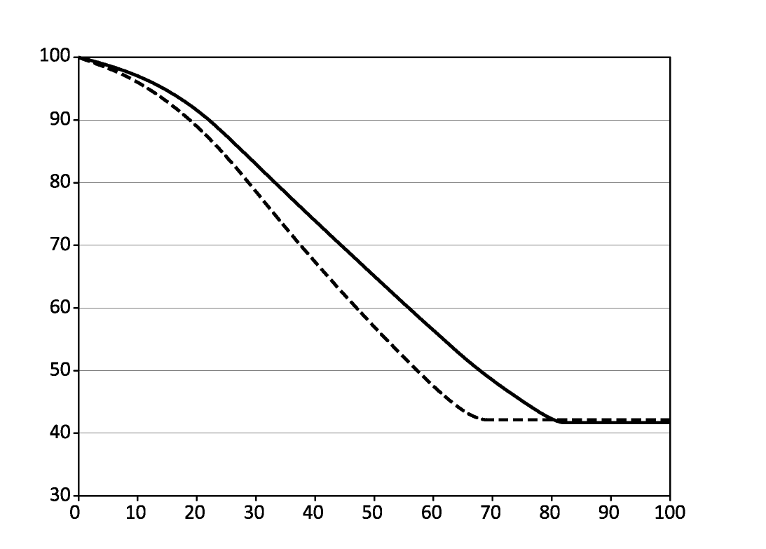

to a method for preparation thereof, and to the cosmetic uses thereof.

The present application relates to the incorporation of pigment into cosmetic compositions. In the sense of the present application, by <<cosmetic composition>> is meant any cosmetic formulation intended to be applied on the skin, hair and/or nails. The formulations may be in the form of a powder, a fluid solution or thickened with thickeners (gel) or aqueous or fatty gelling agents (stick), a water-in-oil or oil-in-water emulsion.

In particular, the invention relates to the incorporation of pigment into aqueous cosmetic compositions. In the sense of the present application, by <<aqueous cosmetic composition>>, is meant a cosmetic composition for which the medium is an aqueous medium, i.e. it comprises from 1 to 95% by mass of water, notably 10 to 70% by mass of water, for example from 20 to 60% by mass of water, said aqueous medium may be a fluid or thickened aqueous solution or an oil-in-water emulsion.

Many cosmetic compositions comprise solids dispersed in a divided state, notably pigments. The more the pigments are dispersed homogeneously in the cosmetic composition, the more homogeneous and intense is the color of the cosmetic composition. The homogeneous dispersion of the pigments in a cosmetic composition is ensured by a step of grinding the pigment in one of the liquid phases of the cosmetic composition.

In order to allow good dispersion of pigment into non-aqueous cosmetic compositions, treatment of the pigments with hydrogenated lecithin has been proposed. Hydrogenated lecithin includes phosphate mono- and di-esters including fatty chains. Once it is deposited on the pigment, hydrogenated lecithin promotes dispersion of the latter in the oily phase. The use of pigments treated with hydrogenated lecithin in a cosmetic composition, the medium of which is oily, therefore gives the possibility of obtaining adequate coloration without any grinding step. However, the pigments treated with hydrogenated lecithin are not easily dispersible in aqueous compositions.

The dispersion of pigments in aqueous cosmetic compositions is more delicate and to this day, only very few treated pigments exist which are easily dispersible in an aqueous medium.

One of the goals of the present invention is to provide a solution for easily and efficiently dispersing a pigment in a cosmetic composition notably an aqueous cosmetic composition.

For this purpose, the present invention provides a pigment composition comprising, in addition to a pigment, an additive making this pigment easily dispersible within aqueous cosmetic compositions.

More specifically, according to a first aspect, the object of the invention is a pigment composition comprising, among other possible components:
a pigment,

an additive of the following formula (I):

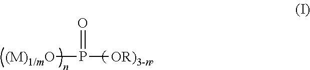

wherein:
n represents 1 or 2,
M represents H or a cation,
m represents 1 when M is H and m represents the valency of the cation when M is a cation,
R represents:
   a group G selected from a saccharide or a group —[CH₂—CHR₁—O]_{q}—R₂ or —[CH₂—CH(CH₂OH)—O]_{q}—R₂ wherein:
      q represents an integer from 1 to 1,000,
      for each unit CH₂—CHR₁—O, R₁ represents independently H or a methyl,
      R₂ represents H or an alkyl comprising from 1 to 3 carbon atoms, and
   a hydrocarbon chain comprising from 1 to 500 carbon atoms substituted with one or several G, phosphate (of formula OPO₃(M)_{2/m}) and/or hydroxyl (OH) groups.

This pigment composition is suitable for introduction into a cosmetic composition such as an aqueous cosmetic composition. It generally appears as a powder comprising the intimately mixed pigment and additive, the additive being generally adsorbed or precipitated at the surface of the pigment.

A pigment composition in the form of a powder is particularly advantageous over a composition in liquid form (suspension, emulsion or solution).

First of all, it does not comprise any liquid phase and is therefore more lightweight. The costs for transport and storage, and a fortiori the cost of the pigment composition are therefore lower.

Further, it does not comprise any liquid phase based on solvents, which may be toxic or flammable.

Finally, a pigment composition as a powder may advantageously be dispersed regardless of the medium of the composition in which dispersion is desired. On the contrary, for a pigment composition in liquid form, it should be ascertained that the liquid phase of the pigment composition and that of the composition into which dispersion is desired, are compatible and may mix together in order to form a homogeneous medium. Further, a pigment composition in liquid form imposes the introduction of an amount of liquid phase which, either by its chemical nature or by its organoleptic properties, may not be desired in the composition in which dispersion is desired. Thus, the possibilities of formulating a pigment composition as a powder are much more extended than those of a pigment composition in liquid form.

It is found that the pigment compositions according to the invention disperse very easily and efficiently in an aqueous medium or in the continuous aqueous phase of an oil-in-water emulsion. Without being bound to a particular theory, the investigations made by the inventors within the scope of the invention give the possibility of putting forward that the hydrophilic group R of the additive of formula (I) would facilitate dispersion of the pigment composition in an aqueous medium.

Moreover, the pigment composition according to the invention advantageously has an improved water retention capacity, relatively to that of the pigment alone. Thus, the pigment composition according to the invention and therefore the cosmetic composition in which it is dispersed, have good moisturizing power. The water retention capacity may notably be demonstrated by tracking the loss of mass over time (thermogravimetry analysis), which may be directly correlated with the evaporation of water, of an aqueous dispersion of a pigment composition according to the invention as compared with that of an aqueous dispersion of the pigment alone.

Without intending to be bound by a particular theory, it seems that this property may be related to the presence, in the chemical formula of the additives of the pigment composition (which are located at the surface of the pigment), of groups which may be engaged into hydrogen bounds (oxygen atom or hydroxyl group for example) and therefore may form hydrogen bonds with water, which would explain the increased water retention capacity.
Additive of formula (I)
The pigment composition comprises an additive of formula (I) as defined above.

The group —[CH₂—CHR₁—O]_{q}—R₂ with R₁ representing H corresponds to a polyethylene glycol (PEG). The group —[CH₂—CHR₁—O]_{q}—R₂ with R₁ representing a methyl corresponds to a polypropylene glycol (PPG). The group —[CH₂—CH(CH₂OH)—O]_{q}—R′ corresponds to a polyglycerol.

Typically q is an integer from 1 to 500, notably from 1 to 100, preferably from 1 to 60.

Preferably, n represents 2 and the additive has the following formula (I′)

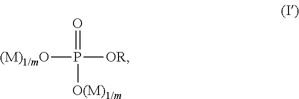

wherein M, m and R are as defined above.

In the sense of the present application, a hydrocarbon chain comprises from 1 to 500 carbon atoms, notably from 1 to 50, typically from 1 to 10 carbon atoms, preferably from 1 to 5 carbon atoms. The hydrocarbon chains may be linear, branched or cyclic. The preferred hydrocarbon chains are alkyl groups (preferably having from 1 to 10 carbon atoms, notably from 1 to 5 carbon atoms, preferably from 1 to 3, such as methyl, ethyl, n-propyl and isopropyl groups), alkenyl groups (preferably having from 2 to 10 carbon atoms, in particular from 2 to 6), aryl groups (preferably having from 6 to 10 carbon atoms), arylalkyl groups (preferably having from 7 to 10 carbon atoms) or alkylaryl groups (preferably having from 7 to 10 carbon atoms). The vinyl group is the preferred alkenyl group. The phenyl group is the preferred aryl.

A saccharide may be a mono- or poly-saccharide. The preferred saccharides are mono- or di-saccharides, in particular mono-saccharides such as glucose, galactose or fructose.

M may notably be an inorganic cation such as Ag³⁺, Al³⁺, Fe³⁺, Fe²⁺, Ag²⁺, Zn²⁺, Sn²⁺, Ca²⁺, Ba²⁺, Ag⁺, Na⁺ or an organic cation, such as diethanolammonium (DEA) (H₃N⁺⁻(CH₂)₂—OH) or a quaternary ammonium.

The following additives of the following formulae (II), (III) or (IV) are particularly adapted to carry out the invention:

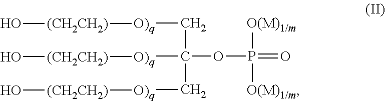

wherein M, m and q are as defined above,
(which corresponds to an additive of formula (I) wherein n represents 2 and R represents an isopropyl hydrocarbon chain, for which each of the carbon atoms is substituted with a group G which represents —[CH₂—CHR₁—O]_{q}—R₂ wherein R₁ and R₂ represent H),

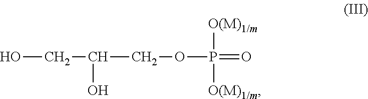

wherein M and m are as defined above,
(which corresponds to an additive of formula (I) in which n represents 2, R represents a group G of formula —[CH₂—CH(CH₂OH)—O]_{q}—R₂ wherein q represents 1 and R₂ represents H),

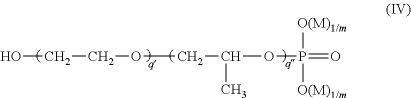

wherein M and m are as defined above and q′ and q″ represent independently an integer from 0 to 1,000, generally from 0 to 500, notably from 0 to 100, preferably from 0 to 60, such that the sum of q′ and q″ represent independently an integer from 1 to 1,000,
(which corresponds to an additive of formula (I) wherein n represents 2, R represents a group G of formula —[CH₂—CHR₁—O]_{q}—R₂ wherein q represents the sum of q′ and q″ and for the first q″ units, R₁ represents a methyl and for the last q′ units, R₁ represents H and R₂ represents H).

The additives of formula (III) (and in particular of formula (III′) as defined below), are particularly advantageous in that the pigment composition comprising them:
1) has very little water absorption, i.e. its water absorption capacity is low. Thus, it is possible to prepare a cosmetic composition comprising such a pigment composition which comprises a strong proportion of pigment composition, therefore a strong pigment proportion, (therefore being advantageously highly colored) while remaining fluid, which is advantageous since highly viscous cosmetic compositions are not generally very pleasant to use.
2) flocculates when it is introduced into a cosmetic composition in the form of a water-in-oil emulsion comprising acrylate copolymers, whereby a cosmetic composition is obtained, comprising the pigment composition encapsulated by the acrylate copolymers. The acrylate copolymers comprise monomer units selected from acrylic acid, ethyl acrylate, methyl methylacrylate, butyl acrylate, or ethylhexyl acrylate. Thus, the cosmetic composition has the white color due to the emulsion but when it is rubbed, for example on the skin, the pigment composition is released from the acrylate copolymers and the cosmetic composition assumes the color of the pigment composition. A cosmetic composition comprising a pigment composition comprising the additive of formula (III) (notably III′)) therefore has the capability of changing color (a so-called <<color changing>> cosmetic composition).
A cosmetic composition comprising:
   a pigment composition, preferably in the form of a water-in-oil emulsion, comprising:
      an additive of formula (III) (preferably (III′) as defined below), and
      a pigment, and
   an acrylate copolymer,
is therefore one of the objects of the present invention.
3) has very strong oil absorption, i.e. its absorption capacity of oils or fats is strong.

Therefore, a cosmetic composition comprising such a pigment composition is not wetted by the sebum of the skin and is particularly suitable for being applied on an oily skin.

The following additives of formula (V) and (VI) are also suitable for carrying out the invention:

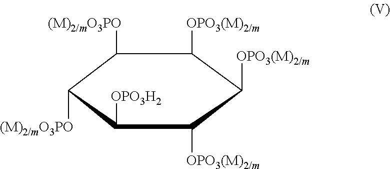

(which corresponds to an additive of formula (I) in which n represents 2, R represents a cyclohexyl hydrocarbon chain substituted in positions 2, 3, 4, 5 and 6 with a phosphate group of formula OPO₃H₂),

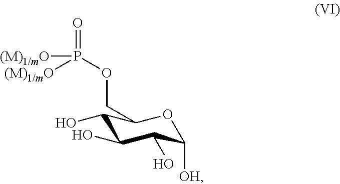

(which corresponds to an additive of formula (I) in which n represents 2, R represents a methyl hydrocarbon chain bound to a glucose group G),
   wherein M and m are as defined above.

The additives of formula (V) (and in particular of formula (V′) as defined below), are particularly advantageous in that the pigment composition comprising them:
1) has better affinity for water than for oil. This pigment composition may therefore be easily introduced into aqueous cosmetic compositions. In particular, it is possible to introduce this pigment composition into a biphasic cosmetic composition, comprising an aqueous phase in which the pigment composition comprising the additive of formula (V) is dispersed, on the one hand and a fatty or oily phase comprising a hydrophobic-treated pigment (obtained by a treatment known in the state of the art, for example with a hydrogenated lecithin additive) on the other hand. Thus, a biphasic cosmetic composition, each of the phases of which has its own color, is obtained. Such a biphasic cosmetic composition may for example be a biphasic foundation.
2) is compatible with anionic polymers of the sodium polyacrylate, carboxymethylcellulose or xanthan gum type customarily used for formulating cosmetic compositions. Now, certain pigments, such as iron oxide, have the drawback of being incompatible with these polymers. By means of the additive of formula (V), it is possible to obtain a cosmetic composition comprising:
   a pigment composition comprising:
      an additive of formula (V) (preferably (V′) as defined below), and
      a pigment, notably iron oxide, and
   an anionic polymer of the sodium polyacrylate, carboxymethylcellulose or xanthan gum type.
The aforementioned cosmetic composition is one of the objects of the present invention.
3) has very low water absorption, i.e. its water absorption capacity is low. Thus, it is possible to prepare a cosmetic composition comprising such a pigment composition which comprises a strong proportion of pigment composition, therefore a strong proportion of pigment (therefore being advantageously highly colored) while remaining fluid, which is advantageous since highly viscous cosmetic compositions are not generally very pleasant to use.
4) has very strong oil absorption, i.e. its absorption capacity of oils or fats is strong. Therefore, a cosmetic composition comprising such a pigment composition is not wetted by the sebum of the skin and is particularly suitable for being applied on an oily skin.
5) has a very small particle size when it is dispersed in an aqueous medium. Thus, not only the pigment composition is instantaneously dispersed when it is introduced into an aqueous medium, but further the pigment composition remains suspended for a long time in the aqueous medium. These effects are particularly advantageous for cosmetic compositions formulated in the form of foam (bath foams, shampoos, toothpaste or shaving foams for example).

The following additives are more preferred:
glycereth-26 phosphate of the following formula (II′):

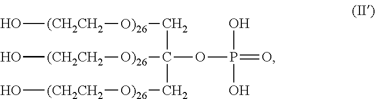

(which corresponds to an additive of formula (II) in which M represents H and m represents 1), this additive being advantageously commercially available, for example from Croda®,
glycerophosphate of the following formula (III′):

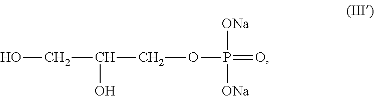

(which corresponds to an additive of formula (II) wherein M represents Na and m represents 1), this additive being advantageously commercially available for example from Dr Paul Lohman®,
diethanolammonium PEG-26 PPG-30 phosphate of the following formula (IV′):

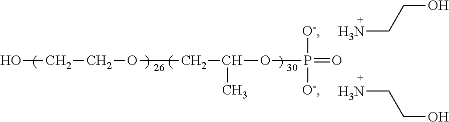

(which corresponds to an additive of formula (IV) wherein M represents a diethanolammonium cation and m represents 1), this additive being advantageously commercially available, for example from Innospec®,
phytic acid of the following formula (V′):

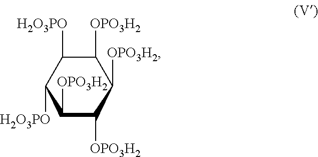

(which corresponds to an additive of formula (V) wherein M represents H and m represents 1), this additive being advantageously commercially available for example from Nutriscience®,
glucose phosphate of the following formula (VI′):

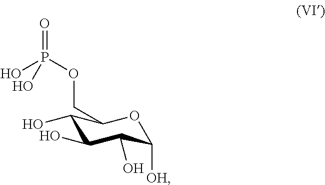

(which corresponds to an additive of formula (VI) wherein M represents H and m represents 1), this additive being advantageously commercially available.
   The pigment

The pigment present in the composition according to the invention may be an organic, inorganic pigment or of an organic-inorganic hybrid nature or a mineral filler. It is typically an inorganic pigment.

As pigments adapted for carrying out the invention, mention may notably be made of titanium dioxide, zinc dioxide, zirconium or cerium oxides, as well as iron or chromium oxides, manganese violet, ultramarine blue, chromium hydrate and ferric blue and mixtures thereof. The preferred mineral pigments are iron oxides, notably red iron oxide, yellow iron oxide, brown iron oxide, black iron oxide, titanium dioxide and mixtures thereof.

In an embodiment, the pigment is an iron oxide, notably red iron oxide, yellow iron oxide, brown iron oxide, black iron oxide and mixtures thereof.

As mineral fillers suitable for carrying out the invention, mention may notably be made of talcum, mica, kaolin, alumina, silica.

Generally, the pigment composition comprises from 0.01% to 20% by weight, typically from 0.1 to 10% by weight, preferably from 0.5 to 5% by weight, in particular of the order of 1% by weight, of additive of formula (I) based on the weight of the combination pigment/additive of formula (I).

The pigment composition according to the invention is hydrophilic and easily disperses in aqueous media, even viscous media, and in the continuous aqueous phase of an oil-in-water emulsion. The inventors have discovered that the use of the pigment composition according to the invention in a cosmetic composition comprising an aqueous medium or an oil-in-water emulsion gives the possibility of obtaining intense and homogeneous coloration. The coloration may for example be measured by spectrocolorimetry and/or spectrophotocolorimetry.

The pigment composition may also contain in addition to the additive of formula (I) and to the pigment, other additives such as polyacrylates or polyacrylic acids, polyethoxylated alkosilanes, surfactants, carboxylic acids comprising ethylene oxide chains, amino acids, chitosan, cellulose and its derivatives (cellulose ether, microcrystalline cellulose, carboxymethylcellulose and hydroxypropyl methylcellulose (HPMC)), sugars such as galactoarabinan or xanthan gum, silica and alumina.

According to a second aspect, the object of the invention is a method for preparing a pigment composition as defined above. This method comprises the following steps:
a) mixing a pigment with an additive as defined above in a solvent,
b) optionally acidification to a pH of less than or equal to 2 and/or addition of a salt after step a),
c) optionally basification to a pH from 3 to 8 after step a) and the optional step b),
d) filtration of the medium obtained after step a) and the optional steps b) and c), optionally followed by one or several steps for rinsing, drying and/or grinding in order to obtain the pigment composition as a solid.

The solvent is generally an aqueous solution preferably water.

Generally, all the steps of the method are carried out at room temperature (of the order of 25° C.) and at atmospheric pressure.

Step a) may be carried out either by mixing the pigment and the additive in the solvent simultaneously, or by preparing a mixture of the additive in a solvent (mixture 1) on the one hand, preparing a mixture of the pigment and of the solvent (mixture 2) and then mixing together both mixtures (mixtures 1+2) on the other hand. No difference in the quality of the pigment composition obtained was observed by varying the addition order of the additive, of the pigment and of the solvent.

During the mixing, the additive is more or less adsorbed on the pigment. It is however preferable that the additive is adsorbed on the pigment. Thus, when adsorption is insufficient, it is advantageous to add a step b), i.e. acidifying the mixture obtained at step a) to a pH of less than or equal to 2 and/or adding a salt to the mixture obtained at step a).

According to the additive and the aqueous medium used:
either the dispersion of a pigment and of an additive of formula (I) in an aqueous medium directly leads to a medium, the pH of which is less than or equal to 2, for example when dispersion of phytic acid and of a pigment in water is prepared, in this case, no acidification of the mixture obtained at step a) is required,
or the dispersion of the pigment and of the additive of formula (I) in an aqueous medium leads to a medium with a pH of more than 2. When the additive is not sufficiently adsorbed on the pigment, a step of acidifying the mixture to a pH of less than or equal to 2 is added.
   This acidification may be carried out by adding an acid (for example hydrochloric acid) to the medium, under conditions known to one skilled in the art.
   Without intending to be bound to a particular theory, at a pH of less than or equal to 2, the phosphate of the additive is in the acid form (M represents H), and the adsorption of the phosphate additive in this form on the pigment is promoted.

The method may also comprise a step b) of adding a salt to the mixture of step a). This salt is typically a salt which may cause precipitation of the additive according to the invention. Generally, the salt is added in a stoichiometric amount of the phosphate groups of the additive.

Without intending to be bound to a particular theory, during the addition of the salt, an exchange of ions would take place between the initial anion of the additive and the anion of the salt, as illustrated in the following scheme:

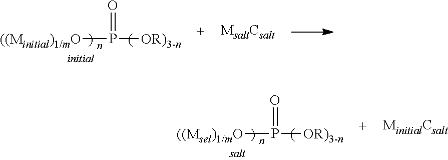

Scheme: Postulated mechanism: Anion exchange between the anion Mᵢₙᵢₜᵢₐₗ of the additive (of charge mᵢₙᵢₜᵢₐₗ) with the anion Mₛₐₗₜ (of charge mₛₐₗₜ) of the added salt, leading to the precipitation of the additive of formula:

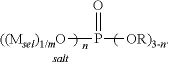

The method may also comprise a step b) consisting of acidifying the mixture to a pH of less than or equal to 2 and of adding a salt to the mixture, the acidification and the addition of a salt may be simultaneous or successive, and in this case the addition of the salt and of the acidification may be performed in any order at step b).

In this embodiment, the method also comprises a subsequent step c) of basification to a pH from 3 to 8, preferably of the order of 5.5. The basification may be carried out by adding a base (for example sodium hydroxide) to the medium, under conditions known to one skilled in the art. Thus, the pH before filtration of the powder is of the order of a pH close to that of the skin, which is suitable for a cosmetic application.

One or several steps of stirring the medium may be added between the steps of the method, typically for 30 minutes to one hour.

At the end of the preparation method, a pigment composition is obtained, which corresponds to a pigment treated with the additive of formula (I). The invention also relates to the pigment compositions which may be obtained by the method defined above.

According to a third aspect, the object of the invention is the use of the pigment composition or of a pigment composition which may be obtained according to the aforementioned method for introduction into a cosmetic composition, notably an aqueous composition (or for preparing a cosmetic composition, notably an aqueous composition), as well as a cosmetic composition comprising the pigment composition.

The cosmetic composition may for example be mascara, foundation, eyeliner, eyeshadow or blush, lipstick, lip gloss, optionally liquid soap, shampoo, conditioner, nail varnish, preferably mascara or foundation. The cosmetic composition may appear as a mono-phasic or bi-phasic lotion, as a water-in-oil or oil-in-water emulsion, gel, cream, or powder.

In an embodiment, the cosmetic composition comprises a pigment composition, the pigment of which is iron oxide, a sequestering agent and a cosmetically acceptable anionic polymer.

The pigments based on iron oxide have the drawback of being incompatible with anionic polymers. Thus, cosmetic compositions comprising iron oxide as a pigment and an anionic polymer are difficult to formulate, since the iron oxide is poorly dispersed therein.

The inventors have discovered that in particular in the presence of a sequestering agent, a pigment composition according to the invention, comprising iron oxide and one of the additives defined above, is very well dispersed in a cosmetic composition comprising a cosmetically acceptable anionic polymer.

It is known that a sequestering agent contributes to the dispersion of the iron oxide in a cosmetic composition comprising a cosmetically acceptable anionic polymer. This being the case, in this embodiment of the invention, the inventors have demonstrated the existence of a synergistic effect of the sequestering agent and of the additive on the dispersion of the iron oxide pigment, since the dispersibility of a pigment composition comprising iron oxide in a cosmetic composition comprising a sequestering agent and an anionic polymer unexpectedly goes beyond the sum of the dispersibilities:
of iron oxide in a cosmetic composition comprising a sequestering agent and an anionic polymer (no pigment composition comprising an additive as defined above) and
of a pigment composition according to the invention comprising iron oxide in a cosmetic composition comprising an anionic polymer and without any sequestering agent.

The sequestering agent is notably selected from phytic acid, citric acid, gluconic acid, pentetic acid, ethylene diamine tetraacetic acid (EDTA), cyclodextrin, salts thereof and a mixture thereof, preferably citric acid or one of its salts.

The salts are preferably formed with an alkaline or earth alkaline metal, preferably an alkaline metal, sodium or potassium salts being more preferred. Mention may notably be made of potassium or sodium citrate, potassium or sodium gluconate, pentasodium pentetate, disodium EDTA salt, trisodium EDTA salt or tetrasodium EDTA salt as a sequestering agent in the form of a salt.

The cosmetically acceptable anionic polymer is typically xanthan gum, carboxymethylcellulose or polyacrylate.

According to a fourth aspect, the object of the invention is a method for preparing a cosmetic composition comprising a pigment comprising a step of dispersing a pigment composition as defined above in an aqueous medium.

Advantageously, starting with the pigment composition as starting product, the method for preparing the cosmetic composition is generally free from grinding step. Therefore it may be applied without a grinder, which is an advantage since grinding equipment is not necessary and the production cost of the cosmetic composition is lower.

According to a fifth aspect, the object of the invention is a cosmetic composition which may be obtained with this method.

The cosmetic composition typically comprises the pigment composition as defined above and at least one cosmetically acceptable excipient.

The examples and the figure hereafter illustrate the invention.

The appended figure illustrates the % ratio of the mass of a 40% aqueous dispersion at instant t over the mass of this aqueous dispersion at t=0 (i.e. at the beginning of the heating, the temperature then being 25° C.) versus time in minutes, either for a 40% aqueous dispersion of a pigment composition of Example 2 Experiment 8 (solid line) or for a 40% aqueous dispersion of red iron oxide (dotted line).

### EXAMPLES

Examples 1 to 4 relate to the preparation of pigment compositions according to the invention and to their introduction into a cosmetic composition of the oil-in-water type.

Examples 5 to 13, 15 and 16 relate to cosmetic compositions according to the invention comprising pigment compositions.

Example 14 relates to the improvement of the water retention capacity of a pigment composition as compared with pigment alone.

Components of the Pigment Composition

Four additives of formula (II′), (III′), (IV′) and (V′) were used in the Examples.

Four pigments were used: black, yellow, red iron oxides, titanium dioxide.

Suppliers of the Various Components Used

TABLE 1
Suppliers of the components used in the Examples.

| Component | Supplier |
|---|---|
| Additive (II′) | Croda |
| Additive (III′) | Dr Paul Lohman |
| Additive (IV′) | Innospec |
| Additive (V′) | Nutriscience |
| Covasop W9775 White | Sensient |
| Covasop W3774 Red | Sensient |
| Black iron oxide | Sensient |
| Yellow iron oxide | Sensient |
| Red iron oxide | Sensient |
| Anatase titanium dioxide | Sensient |
| Rutile titanium dioxide | Sensient |
| UNIPURE WHITE LC 981 HLC | Sensient |
| UNIPURE RED LC 381 HLC | Sensient |
| AlCl₃ 28% | Merck |
| Zn(CH₃COO)₂ 20% | Merck |
| SnCl₂, 15%, in 37% hydrochloric acid | Merck |
| Covathick 2009 | Sensient |
| Natpure SF | Sensient |
| Submica FL | Sensient |
| Covacryl MS11 | Sensient |
| Submica M | Sensient |
| DC 2502 cosmetic fluid | Dow Corning |
| Silamer | Sensient |
| Covacryl SJ5 | Sensient |
| Covacryl P12 | Sensient |
| Covalip 25 | Sensient |
| Macamat wax | Sensient |
| Covagloss | Sensient |
| Covabead LH 85 | Sensient |
| Covafluid FS | Sensient |
| Covamat | Sensient |
| Bentone 38 | elementis |
| Component | Supplier |
| Additive (II′) | Croda |
| Additive (III′) | Dr Paul Lohman |
| Additive (IV′) | Innospec |
| Additive (V′) | Nutriscience |
| Covasop W9775 White | Sensient |
| Covasop W3774 Red | Sensient |
| Black iron oxide | Sensient |
| Yellow iron oxide | Sensient |
| Red iron oxide | Sensient |
| Anatase titanium dioxide | Sensient |
| Rutile titanium dioxide | Sensient |
| AlCl₃ 28% | Merck |
| Zn(CH₃COO)₂ 20% | Merck |
| SnCl₂, 15%, in 37% hydrochloric acid | Merck |

### I. Preparation of Pigment Compositions and Introduction into an Aqueous Cosmetic Composition of the Oil-in-Water Emulsion Type

Methods for Preparation of Pigment Compositions:

### Method A:

mixing water and additive (mixture 1),
mixing the pigment and water (mixture 2),
mixing both mixtures (mixture 1+2),
adding the acid down to a pH of 2,
stirring for one hour,
adding the base up to pH 5.5,
stirring for 30 minutes,
filtering, rinsing with water, drying, grinding.

### Method B:

mixing water, additive and pigment,
stirring for 30 minutes,
adding the salt,
stirring for 30 minutes,
filtering, rinsing with water, drying, grinding.

### Method C:

mixing water, additive and pigment,
adding the salt,
stirring for 30 minutes,
adding the base up to pH 5.5,
stirring for 30 minutes,
filtering, rinsing with water, drying, grinding.

### Method D:

mixing water, additive and pigment,
adding with the acid down to a pH of 2,
stirring for one hour,
adding the base up to pH 5.5,
stirring for 30 minutes,
filtering, rinsing with water, drying, grinding.

### Method E:

mixing water, additive and pigment,
stirring for 1 hour,
filtering, rinsing with water, drying, grinding.

### Method F:

mixing with water, additive and pigment,
stirring for 1 hour,
filtering, no rinsing, drying, grinding.

### Method G:

mixing water, additive and pigment,
stirring for one hour,
adding the acid down to a pH of 2,
stirring for one hour,
adding the base up to pH 5.5,
stirring for 30 minutes,
filtering, rinsing with water, drying, grinding.

### Method H:

mixing water, additive and pigment,
stirring for one hour,
adding the acid down to a pH of 2,
stirring for one hour,
adding the base up to pH 5.5,
stirring for 30 minutes,
filtering, rinsing with water, drying, no grinding, obtaining the pigment as a granule.

### Method I:

mixing water, additive and pigment,
stirring for one hour,
adding the base up to a pH of 5.5,
stirring for 30 minutes,
filtering, rinsing with water, drying, grinding.

Introduction of the Pigment Composition into an Oil-in-Water Emulsion

After preparation of the pigment composition, the latter was introduced into an aqueous cosmetic composition of the oil-in-water emulsion type prepared as follows:
heating a mixture A to 70° C. consisting in:
   48.3% by mass of an oil-in-water base emulsion BASE O/W 097 (Sensient®),
   41.4% by mass of Fluid Mineral Oil (Sensient®), and
   10.3% by mass of Polysorbate 60 (Sensient®),
heating a mixture B to 70° C. consisting in:
   0.7% by mass of Phenonip (Clariant) (preservative),
   99.3% by mass of water.
introducing the mixture B into the mixture A at 70° C. with stirring, in order to obtain an A/B 40/60 mixture and then cooling the emulsion obtained at room temperature,
adding 5% by mass of Covasop W9775 White (Sensient®) or 3% by mass of Covasop W3774 Red (Sensient®) according to the pigment used in the pigment composition and 2% by mass of pigment composition.
the thereby obtained preparation was mixed for 2 minutes with a spatula in order to obtain a colored aqueous cosmetic composition.

The introduction of the pigment composition into the cosmetic composition does not comprise any grinding step.

Covasop corresponds to a pigment dispersion in propylene glycol. It allows degradation of the color of the mixture [emulsion/pigment composition] in order to further reveal the colorimetric differences. When the pigment composition comprises a black, red or yellow iron oxide pigment, the dispersion used is Covasop W9775 White (Sensient®) containing titanium dioxide. When the pigment composition comprises titanium dioxide, the dispersion used is Covasop W3774 Red containing red iron oxide.

Colorimetric Test

The apparatus used is a Minolta Spectrophotometer CM-3300d, and allows measurement of the color in the Lab reference system. The measurement of the color of a colored aqueous cosmetic composition containing the treated pigment relatively to the measurement of the color of a colored aqueous cosmetic composition containing the untreated pigment, called a reference, gives the possibility of obtaining color differences, ΔL* being the luminosity difference, Δa* the difference in the red/green hues and Δb* the difference in the yellow/blue hues.

The references used for calculating color differences were prepared by using the same procedure as the one described above in <<Introduction of the pigment composition in an oil-in-water emulsion>>, except that 2% by mass of reference pigment (pure pigment without any additive, this pigment being the same as the one used in the pigment composition) were added instead of the pigment composition in the last step.

### Example 1

### Preparation of a Pigment Composition Comprising Glycereth-26 Phosphate (Additive of Formula (II′)) and Introduction into an Aqueous Cosmetic Composition of the Oil-in-Water Emulsion Type

### A. With Black Iron Oxide as a Pigment

### a) Influence of the Method for Preparing the Pigment Composition

TABLE 2
Colorimetric results for an oil-in-water emulsion comprising a pigment
composition [black iron oxide/additive of formula (II′)] as compared with an oil-in-water
emulsion comprising untreated black iron oxide (reference).

| Experiment | | Ref^{ce} | 1 | 2 | 3 | 4 | 5 | 6 | 7 |
|---|---|---|---|---|---|---|---|---|---|
| Method followed | | — | A | B | C | D | E | F | G |
| Component | Black | 100% | 47.5 | 95 | 95 | 47.5 | 95 | 95 | 47.5 |
| | iron | | | | | | | | |
| | oxide (g) | | | | | | | | |
| | Water | — | 400 | 400 | 400 | 500 | 1,000 | 1,000 | 500 |
| | (g) | | | | | | | | |
| | Additive | — | 2.5 | 5 | 5 | 2.5 | 2.5 | 2.5 | 2.5 |
| | of | | | | | | | | |
| | formula | | | | | | | | |
| | (II′) (g) | | | | | | | | |
| | Water (if | — | 97.3 | — | — | 0.84 | — | — | 0.96 |
| | mixture | | | | | | | | |
| | 2 exists) | | | | | | | | |
| | (g) | | | | | | | | |
| | 6N HCl | — | 3 | — | — | — | — | — | — |
| | (g) | | | | | | | | |
| | AlCl₃ | — | — | — | — | — | — | — | — |
| | 28% (g) | | | | | | | | |
| | Zn(CH₃ | — | — | 4.5 | — | — | — | — | — |
| | COO)₂ | | | | | | | | |
| | 20% (g) | | | | | | | | |
| | SnCl₂ | — | — | — | 6 | — | — | — | — |
| | 15% in | | | | | | | | |
| | 37% | | | | | | | | |
| | HCl (g) | | | | | | | | |
| | NaOH | — | 1.3 g | — | 3.03 | 0.62 | — | — | 0.74 |
| | 30.5% | | and | | | | | | |
| | (g) | | then | | | | | | |
| | | | 1.25 g | | | | | | |
| Final pH | | ND | 7.3 | 5.0 | 5.5 | 5.61 | ND | ND | 5.84 |
| wt % of additive in | | 0 | 5 | 5 | 5 | 5 | 5 | 5 | 5 |
| the obtained | | | | | | | | | |
| pigment | | | | | | | | | |
| composition | | | | | | | | | |
| Colorimetry | ΔL* | L* = | −12.70 | −10.30 | −10.55 | −8.97 | −11.74 | −11.65 | −15.82 |
| Results | | 52.18 | | | | | | | |
| | Δa* | a* = | 0.21 | 0.18 | 0.23 | 0.09 | 0.22 | 0.21 | 0.31 |
| | | −1.05 | | | | | | | |
| | Δb* | b* = | −0.54 | −0.46 | −0.28 | −0.31 | −0.36 | −0.31 | −0.40 |
| | | −3.01 | | | | | | | |

As a white pigment dispersion is added to the emulsion in order to attenuate the color (cf above), the values of ΔL* (luminosity, black and white) are especially those which are important and which indicate that the obtained emulsion is more or less black. A negative ΔL* is synonymous with a blacker coloration than the reference, and all the blacker since the absolute value of the ΔL* is significant. The more the emulsion is black (more intense black color) the better the iron oxide is dispersed in the emulsion.

The results illustrated in Table 2 show that regardless of the operating procedure used for preparing the pigment composition, the pigment composition is better dispersed in an oil-in-water emulsion than the untreated pigment alone (reference).

### b) Influence of the Additive Percentage in the Pigment Composition

TABLE 3
Colorimetric results for an oil-in-water emulsion comprising a pigment
composition [black iron oxides/additive of formula (II′)] as compared with an oil-in-water
emulsion comprising untreated black iron oxide (reference).

| Experiment | | Ref^{ce} | 8 | 10 | 11 | 12 | 13 |
|---|---|---|---|---|---|---|---|
| Method followed | | — | G | G | G | G | G |
| Component | Black iron | 100% | 49.5 | 49 | 48.5 | 47.5 | 46.5 |
| | oxide (g) | | | | | | |
| | Water (g) | — | 500 | 500 | 500 | 500 | 500 |
| | Additive of | — | 0.5 | 1 | 1.5 | 2.5 | 3.5 |
| | formula (II′) | | | | | | |
| | (g) | | | | | | |
| | 6N HCl (g) | — | 0.64 | 1.09 | 0.82 | 0.96 | 0.60 |
| | NaOH | — | 0.39 | 0.73 | 0.63 | 0.74 | 0.51 |
| | 30.5% (g) | | | | | | |
| Final pH | | ND | 6.13 | 6.7 | 6 | 5.84 | 5.78 |
| wt % of additive in the | | 0 | 1 | 2 | 3 | 5 | 7 |
| obtained pigment | | | | | | | |
| composition | | | | | | | |
| Colorimetric | ΔL* | L* = 52.18 | −14.25 | −12.79 | −16.17 | −15.82 | −16.48 |
| results | Δa* | a* = −1.05 | 0.23 | 0.26 | 0.27 | 0.31 | 0.26 |
| | Δb* | b* = −3.01 | −0.61 | −0.34 | −0.55 | −0.40 | −0.64 |

The results illustrated in Table 3 show that the use of a pigment composition comprising from 1 to 6 wt % of additive gives the possibility of obtaining a better dispersion in an oil-in-water emulsion than with the pigment alone (reference).

B. With other Pigments

TABLE 4
Colorimetric results for an oil-in-water emulsion comprising a pigment
composition [pigment/additive of formula (II′)] as compared with an oil-in-water emulsion
comprising an untreated pigment (reference).

| | | Pigment | | | | | |
|---|---|---|---|---|---|---|---|
| | | | | | | Rutile titanium | |
| | | Yellow iron oxide | | Red iron oxide | | dioxide | |
| Experiment | | Ref^{ce} | 14 | Ref^{ce} | 15 | Ref^{ce} | 17 |
| Method followed | | — | G | — | G | — | G |
| Component | Pigment | 100% | 49.5 | 100% | 49.5 | 100% | 49.5 |
| | (g) | | | | | | |
| | Water (g) | — | 500 | — | 500 | — | 500 |
| | Additive of | — | 0.5 | — | 0.5 | — | 0.5 |
| | formula | | | | | | |
| | (II′) (g) | | | | | | |
| | 6N HCl (g) | — | 1.12 | — | 1.3 | — | 1.37 |
| | NaOH | — | 0.97 | — | 0.9 | — | 0.84 |
| | 30.5% (g) | | | | | | |
| Final pH | | ND | 5.77 | ND | 5.9 | ND | 5.83 |
| wt % of additive in the | | 0% | 1 | 0% | 1 | 0% | 1 |
| obtained pigment | | | | | | | |
| composition | | | | | | | |
| Colorimetric | ΔL* | L* = 78.30 | −6.44 | L* = 66.46 | −8.43 | L* = 58.24 | 5.51 |
| results | Δa* | a* = 6.77 | 5.75 | a* = 19.10 | 8.64 | a* = 27.24 | −3.44 |
| | Δb* | b* = 28.93 | 16.91 | b* = 11.30 | 9.28 | b* = −1.45 | 0.19 |

The results of Table 4 show that the additive used in the pigment composition is also efficient for other pigments.

### Example 2

### Preparation of a Pigment Composition Comprising Sodium Glycerophosphate (Additive of Formula (III′)) and Introduction into an Aqueous Cosmetic Composition of the Oil-in-Water Emulsion Type

### A. With Black Iron Oxide as a Pigment

TABLE 5
Colorimetric results for an oil-in-water emulsion comprising a pigment
composition [black iron oxide/additive of formula (III′)] as compared with an oil-in-water
emulsion comprising non-additived black iron oxide (reference).

| Experiment | | Ref^{ce} | 1 | 2 | 3 | 4 | 5 | 6 |
|---|---|---|---|---|---|---|---|---|
| Method followed | | — | D | G | G | G | G | G |
| Component | Black | 100% | 47.5 | 49.0 | 48.5 | 47.5 | 47.0 | 95 |
| | iron | | | | | | | |
| | oxide (g) | | | | | | | |
| | Water | — | 400 | 500 | 500 | 500 | 500 | 1,000 |
| | (g) | | | | | | | |
| | Additive | — | 2.5 | 1 | 1.5 | 2.5 | 3 | 2.5 |
| | of | | | | | | | |
| | formula | | | | | | | |
| | (III′) (g) | | | | | | | |
| | 6N HCl | — | 2.75 | 1.99 | 2.33 | 3.09 | | |
| | (g) | | | | | | | |
| | NaOH | — | 1.1 | 0.70 | 0.75 | 0.87 | | |
| | 30.5% | | | | | | | |
| | (g) | | | | | | | |
| Final pH | | ND | 5.5 | 5.25 | 5.26 | 5.5 | | |
| wt % of additive in the | | 0 | 5 | 2 | 3 | 5 | 6 | 7 |
| obtained pigment | | | | | | | | |
| composition | | | | | | | | |
| Colorimetric | ΔL* | L* = 52.18 | −7.19 | −9.10 | −8.62 | −14.85 | −18.65 | −13.63 |
| results | Δa* | a* = −1.05 | 0.23 | 0.23 | 0.06 | 0.25 | 0.45 | 0.15 |
| | Δb* | b* = −3.01 | −0.11 | −0.10 | −0.51 | −0.70 | −0.53 | −0.81 |

The results illustrated in Table 5 show that this additive is also efficient for improving the dispersion of the powder in an oil-in-water emulsion as compared with the pigment used alone (reference).

### B. With Other Pigments

TABLE 6
Colorimetric results for an oil-in-water emulsion comprising a pigment
composition [pigment/additive of formula (III′)] as compared with an oil-in-water emulsion
comprising an untreated pigment (reference).

| | | Pigment | | | | | |
|---|---|---|---|---|---|---|---|
| | | Yellow iron | | | | Rutile titanium | |
| | | oxide | | Red iron oxide | | dioxide | |
| Experiment | | Ref^{ce} | 7 | Ref^{ce} | 8 | Ref^{ce} | 10 |
| Method followed | | — | G | — | G | — | G |
| Component | Pigment (g) | 100% | 49.5 | 100% | 49.5 | 100% | 49.5 |
| | Water (g) | — | 500 | — | 500 | — | 500 |
| | Additive of | — | 0.5 | — | 0.5 | — | 0.5 |
| | formula (III′) (g) | | | | | | |
| | 6N HCl (g) | — | 1.71 | — | 1.77 | — | 1.89 |
| | NaOH 30.5% (g) | — | 1.08 | — | 0.89 | — | 0.14 |
| Final pH | | ND | 5.28 | ND | 5.66 | ND | 6.22 |
| wt % of additive in the obtained | | 0 | 1 | 0 | 1 | 0 | 1 |
| pigment composition | | | | | | | |
| Colorimetric | ΔL* | L* = 78.30 | −6.22 | L* = 66.44 | −12.27 | L* = 58.24 | 6.17 |
| results | Δa* | a* = 6.77 | 5.51 | a* = 19.10 | 9.62 | a* = 27.24 | −4.01 |
| | Δb* | b* = 28.93 | −16.32 | b* = 11.33 | 7.66 | b* = −1.45 | 0.95 |

The results of Table 6 show that the additive used in the pigment composition is also efficient for other pigments.

### Example 3

### Preparation of a Pigment Composition Comprising PEG-26 PPG-30 DEA Phosphate (Additive of Formula (IV′)) and Introduction into an Aqueous Cosmetic Composition of the Oil-in-Water Emulsion Type

### A. With Black Iron Oxide as Pigment

TABLE 7
Colorimetric results for an oil-in-water emulsion comprising a pigment
composition [black iron oxide/additive of formula (IV′)] as compared with an oil-in-water
emulsion comprising untreated black iron oxide (reference).

| Experiment | | Ref^{ce} | 1 | 2 | 3 | 4 | 5 | 6 |
|---|---|---|---|---|---|---|---|---|
| Method followed | | — | G | G | G | G | G | G |
| Component | Black iron | 100% | 49.5 | 48.5 | 48.0 | 47.5 | 47.0 | 46.5 |
| | oxide (g) | | | | | | | |
| | Water (g) | — | 500 | 500 | 500 | 400 | 500 | 500 |
| | Additive of | — | 0.5 | 1.5 | 2 | 2.5 | 3 | 3.5 |
| | formula (IV′) | | | | | | | |
| | (g) | | | | | | | |
| | 6N HCl (g) | — | 1.55 | 0.65 | 0.73 | 0.86 | 0.87 | 0.86 |
| | NaOH 30.5% | — | 0.96 | 0.46 | 0.50 | 0.55 | 0.71 | 0.63 |
| | (g) | | | | | | | |
| Final pH | | ND | 6.50 | 6.40 | 6.26 | 5.5 | 5.97 | 5.47 |
| wt % of additive | | 0% | 1 | 3 | 4 | 5 | 6 | 7 |
| in the obtained | | | | | | | | |
| pigment composition | | | | | | | | |
| Colorimetric | ΔL* | L* = 52.18 | −14.45 | −14.41 | −14.81 | −20.17 | −13.90 | −14.91 |
| results | Δa* | a* = −1.05 | 0.30 | 0.29 | 0.34 | 0.37 | 0.19 | 0.23 |
| | Δb* | b* = −3.01 | −0.92 | −0.56 | −0.35 | −0.89 | −0.62 | −0.66 |

The results illustrated in Table 7 show that this additive is also efficient for improving the dispersion of the powder in an oil-in-water emulsion as compared with the pigment used alone (reference).

### B. With Other Pigments

TABLE 8
Colorimetric results for an oil-in-water emulsion comprising a pigment
composition [pigment/additive of formula (IV′)] as compared with an oil-in-water emulsion
comprised an untreated pigment (reference).

| | | Pigment | | | | | | | |
|---|---|---|---|---|---|---|---|---|---|
| | | Yellow iron | | Red iron | | Titanium | | | |
| | | oxide | | oxide | | dioxide | | TiO₂ LC987 | |
| Experiment | | Ref^{ce} | 7 | Ref^{ce} | 8 | Ref^{ce} | 10 | Ref^{ce} | 11 |
| Following method | | — | G | — | G | — | G | — | G |
| Component | Pigment (g) | 100% | 49.5 | 100% | 49.5 | 100% | 49.5 | 100% | 49.5 |
| | Water (g) | — | 500 | — | 500 | — | 500 | — | 500 |
| | Additive of | — | 0.54 | — | 0.54 | — | 0.54 | — | 0.54 |
| | formula (IV′) (g) | | | | | | | | |
| | 6N HCl (g) | — | 1.20 | — | 1.15 | — | 1.25 | — | 1.30 |
| | NaOH 30.5% (g) | — | 0.93 | — | 0.79 | — | 0.86 | — | 0.81 |
| Final pH | | ND | 5.31 | ND | 5.93 | ND | 5.60 | ND | 5.82 |
| wt % of additive in the | | 0 | 1 | 0 | 1 | 0 | 1 | 0 | 1 |
| obtained pigment | | | | | | | | | |
| composition | | | | | | | | | |
| Colorimetric | ΔL* | L* = 78.30 | −5.28 | L* = 66.46 | −6.85 | L* = 32.67 | 5.00 | L* = 58.24 | 10.78 |
| results | Δa* | a* = 6.77 | 4.38 | a* = 19.10 | 5.66 | a* = 0.36 | −0.83 | a* = 27.24 | −7.41 |
| | Δb* | b* = 28.93 | 12.33 | b* = 11.33 | 5.12 | b* = −2.56 | −0.56 | b* = −1.45 | 1.26 |

The results of Table 8 show that the additive used in the pigment composition is also efficient for other pigments.

### Example 4

### Preparation of a Pigment Composition Comprising Phytic Acid (Additive of Formula (V′)) and Introduction into an Aqueous Cosmetic Composition of the Oil-in-Water Emulsion Type

### A. With Black Iron Oxide as a Pigment

TABLE 9
Colorimetric results for an oil-in-water emulsion comprising a pigment
composition [black iron oxide/additive of formula (V′)] as compared with an oil-in-water
emulsion comprising untreated black iron oxide (reference).

| Experiment | | Ref^{ce} | 1 | 2 | 3 | 4 | 5 | 6 | 7 |
|---|---|---|---|---|---|---|---|---|---|
| Method followed | | — | I | I | I | I | I | I | I |
| Component | Black | 100% | 49.5 | 49.0 | 48.5 | 48.0 | 47.5 | 47.0 | 46.0 |
| | iron | | | | | | | | |
| | oxide (g) | | | | | | | | |
| | Water | — | 500 | 500 | 500 | 500 | 500 | 500 | 500 |
| | (g) | | | | | | | | |
| | Additive | — | 1 | 2 | 3 | 4 | 5 | 6 | 7 |
| | of | | | | | | | | |
| | formula | | | | | | | | |
| | (V′) (g) | | | | | | | | |
| | at 50% | | | | | | | | |
| | NaOH | — | 0.44 | 1.02 | 0.73 | 2.32 | 2.85 | 3.62 | 4.36 |
| | 30.5% | | | | | | | | |
| | (g) | | | | | | | | |
| Final pH | | ND | 5.81 | 5.53 | 5.69 | 5.45 | 5.5 | 5.45 | 5.55 |
| wt % of additive in | | 0% | 1 | 2 | 3 | 4 | 5 | 6 | 7 |
| the obtained | | | | | | | | | |
| pigment | | | | | | | | | |
| composition | | | | | | | | | |
| Colorimetric | ΔL* | L* = 52.18 | −14.28 | −12.16 | −12.94 | −13.32 | −14.63 | −16.05 | −12.31 |
| results | Δa* | a* = −1.05 | 0.14 | 0.13 | 0.15 | 0.15 | 0.09 | 0.16 | 0.13 |
| | Δb* | b* = −3.01 | −0.88 | −0.56 | −0.6 | −0.6 | −0.95 | −0.99 | −0.78 |

The results illustrated in Table 9 show that this additive is also efficient for improving dispersion of the powder into an oil-in-water emulsion as compared with the pigment used alone (untreated) (reference).

### B. With Other Pigments

TABLE 10
Colorimetric results for an oil-in-water emulsion comprising a pigment
composition [pigment/additive of formula (V′)] as compared with an oil-in-water emulsion
comprising an untreated pigment (reference).

| | | Pigment | | | | | | | |
|---|---|---|---|---|---|---|---|---|---|
| | | | | | | Anatase | | Rutile | |
| | | Yellow iron | | Red iron | | titanium | | titanium | |
| | | oxide | | oxide | | dioxide | | dioxide | |
| Experiment | | Ref^{ce} | 8 | Ref^{ce} | 9 | Ref^{ce} | 10 | Ref^{ce} | 11 |
| Method followed | | — | I | — | I | — | I | — | I |
| Component | Pigment | 100% | 49.5 | 100% | 49.5 | 100% | 49.5 | 100% | 49.5 |
| | (g) | | | | | | | | |
| | Water (g) | — | 500 | — | 500 | — | 500 | — | 500 |
| | Additive of | — | 1 | — | 1 | — | 1 | — | 1 |
| | formula | | | | | | | | |
| | (V′) (g) | | | | | | | | |
| | NaOH | — | 0.65 | — | 0.56 | — | 0.68 | — | 0.52 |
| | 30.5% (g) | | | | | | | | |
| Final pH | | ND | 5.35 | ND | 5.68 | ND | 5.34 | ND | 5.95 |
| wt % of additive in the | | 0 | 1 | 0 | 1 | 0 | 1 | 0 | 1 |
| obtained pigment | | | | | | | | | |
| composition | | | | | | | | | |
| Colorimetric | ΔL* | L* = 78.30 | −5.15 | L* = 66.46 | −13.71 | L* = 32.67 | 2.60 | L* = 58.24 | 8.26 |
| results | Δa* | a* = 6.77 | 4.82 | a* = 19.10 | 12.66 | a* = 0.36 | −0.26 | a* = 27.24 | −5.41 |
| | Δb* | b* = 28.93 | 14.67 | b* = 11.33 | 12.08 | b* = −2.56 | −0.66 | b* = −1.45 | 1.13 |

The results of Table 10 show that the additive used in the pigment composition is also efficient for other pigments.

Thus, Examples 1 to 4 demonstrate that the pigment compositions according to the invention are easily dispersed in a cosmetic composition of the oil-in-water type, without any grinding step being necessary for incorporating the pigment composition during the preparation of the cosmetic composition.

### II. Cosmetic Compositions Comprising the Pigment Compositions

### Example 5

### Biphasic (Mineral Oil/Aqueous Phase) Foundation Comprising a Pigment Composition Comprising the Additive of Formula (V′)

TABLE 11
Composition of the biphasic foundation

| | Mass |
|---|---|
| Ingredient | (g) |
| Demineralized water | 49.00 |
| Pigment composition containing yellow iron oxide and the additive | 0.25 |
| of formula (V′) as described in Example 4 Experiment 8 | |
| Pigment composition containing titanium dioxide and the additive | 0.75 |
| of formula (V′) as described in Example 4 Experiment 10 | |
| Mineral Oil | 49.00 |
| UNIPURE RED LC 381 HLC | 0.15 |
| UNIPURE WHITE LC 981 HLC | 0.85 |

The pigment compositions containing the additive of formula (V′) were introduced into water. Unipure Red LC381 HLC (hydrophobic red iron oxide) and Unipure White LC981 HLC (hydrophobic titanium dioxide) were introduced in the mineral oil. The fatty phase was then poured onto the aqueous phase in order to form a biphasic medium. The obtained biphasic foundation comprises the pigment composition according to the invention in the aqueous phase while Unipure Red LC381 HLC and Unipure White LC981 HLC are in the oily phase. The foundation should be stirred before use.

This example illustrates the affinity for water of the pigment composition containing phytic acid (additive of formula (V′)) in a cosmetic composition of the biphasic foundation type.

### Example 6

### O/W Foundation Comprising a Pigment Composition Comprising the Additive of Formula (III′)

TABLE 12
Composition of the O/W foundation

| | | Mass |
|---|---|---|
| | Ingredients | (g) |
| A | Macadamia Ternifolia Seed Oil | 25.00 |
| B | Demineralized water | 53.70 |
| | Preservative | 0.30 |
| C | Glycerol | 2.00 |
| | Covathick 2009: Microcrystalline cellulose (and) Cellulose | 0.50 |
| | Gum (anionic polymer) | |
| D | Pigment composition containing titanium dioxide and the | 6.50 |
| | additive of formula (III′) as described in Example 2 | |
| | Experiment 10 | |
| | Pigment composition containing yellow iron oxide and the | 1.41 |
| | additive of formula (III′) as described in Example 2 | |
| | Experiment 7 | |
| | Pigment composition containing red iron oxide and the | 0.47 |
| | additive of formula (III′) as described in Example 2 | |
| | Experiment 8 | |
| | Pigment composition containing black iron oxide and the | 0.12 |
| | additive of formula (III′) as described in Example 2 | |
| | Experiment 2 | |
| E | Natpure SF: Sucrose Stearate (and) Sucrose Distearate | 5.00 |
| F | Submica FL: Mica | 5.00 |

Covathick 2009 was dispersed in glycerol and then introduced into water with stirring, and the four premixed pigment compositions were then introduced without grinding into the gelled aqueous phase. Natpure SF was introduced into the aqueous phase containing the pigment compositions. The aqueous phase containing the pigment compositions was heated to 60° C. and introduced with stirring into macadamia oil heated to 60° C. Finally, Submica FL was added to the final emulsion.

This example illustrates the compatibility of the pigment composition containing glycerophosphate (additive of formula (III′)) with the anionic polymers in a cosmetic composition of the oil-in-water foundation emulsion type.

### Examples 7 to 9

Examples 7 to 9 illustrate the property of very low water absorption of the pigment composition containing the additive of formula (V′) in a cosmetic composition of the mascara, foundation and eyeliner type.

### Example 7

### O/W Mascara Comprising a Pigment Composition Comprising the Additive of Formula (V′)

TABLE 13
Composition of the O/W mascara

| | | Mass |
|---|---|---|
| | Ingredients | (g) |
| A | Base RW136: Stearic acid (and) Cetheareth-25 (and) PPG-2 | 21.00 |
| | Stearate (and) Mineral Oil (and) Synthetic Beeswax (and) | |
| | Cera Carnauba (and) Cetyl alcohol | |
| | Dimethicone 350 cst | 0.50 |
| B | Demineralized water | 22.00 |
| | Preservative | 0.45 |
| C | Thickagent LC: Xanthan Gum (and) Hectorite (and) Cellulose | 0.30 |
| D | Demineralized water | 21.8 |
| | Triethanolamine | 1.30 |
| E | Cyclopentasiloxane | 4.00 |
| | Pigment composition containing black iron oxide and the | 12.00 |
| | additive of formula (V′) as described in Example 4 | |
| | Experiment 1 | |
| | Covacryl P12: Acrylates Copolymer | 16.65 |

The oily phase consisting of the base RW136 and of dimethicone was heated to 85° C. The water and the preservatives were also heated to 85° C., and the thickener based on xanthan gum was introduced into this aqueous phase. This aqueous phase was introduced with stirring into the oily phase. Next, the water containing triethanolamine was heated to 70° C. and added to the previous mixture. Cyclopentasiloxane was then introduced into the emulsion. The pigment composition was then introduced without any grinding. Next, the acrylic copolymer was added to the emulsion.

### Example 8

### Watercolor Foundation Comprising a Pigment Composition Comprising the Additive of Formula (V′)

TABLE 14
Composition of the watercolor foundation

| | Mass |
|---|---|
| Ingredients | (g) |
| Pigment composition containing titanium dioxide and the additive | 39.74 |
| of formula (V′) as described in Example 4 Experiment 10 | |
| Pigment composition containing black iron oxide and the additive | 1.34 |
| of formula (V′) as described in Example 4 Experiment 1 | |
| Pigment composition containing red iron oxide and the additive | 2.42 |
| of formula (V′) as described in Example 4 Experiment 9 | |
| Pigment composition containing yellow iron oxide and the additive | 10.10 |
| of formula (V′) as described in Example 4 Experiment 8 | |
| Pigment composition containing talcum and the additive of formula | 17.90 |
| (V′) | |
| Glycerol | 17.90 |
| Demineralized water | 10.60 |

All the ingredients were mixed and cast into a cup. The dispersion was then dried at 60° C. The thereby obtained solid foundation is used with an applicator moistened with water.

### Example 9

### White Eyeliner Comprising a Pigment Composition Comprising the Additive of Formula (V′)

TABLE 15
Composition of the white eyeliner

| | | Mass |
|---|---|---|
| | Ingredients | (g) |
| A | Monopropylene glycol | 1.4 |
| | Thickagent LC: Xanthan Gum (and) Hectorite (and) Cellulose | 0.7 |
| | Demineralized water | 22.60 |
| B | Demineralized water | 15.00 |
| | Preservative | 0.30 |
| C | Pigment composition containing titanium dioxide and the | 30.00 |
| | additive of formula (V′) as described in Example 4 | |
| | Experiment 10 | |
| | Glycerol | 15.00 |
| D | Covacryl MS11: acrylate copolymer | 15.00 |

The Thickagent LC was dispersed in monopropylene glycol and then introduced with stirring into the water. The obtained gel was diluted in phase B. The pigment composition was dispersed in glycerol and then introduced into the gel. Covavryl MS11 was introduced.

Examples 7 to 9 above illustrate the property of very low water absorption of the pigment composition containing the additive of formula (V′) in a cosmetic composition of the mascara, foundation and eyeliner type.

### Example 10

### O/W Cosmetic Composition of the “Color Changing” Type Comprising a Pigment Composition Comprising the Additive of Formula (III′)

TABLE 16
Composition of the cosmetic composition of the [symbol] color changing [symbol] type

| | | Mass |
|---|---|---|
| | Ingredients | (g) |
| A | Silamer: Phenyl Trimethicone (and) Cetyl PEG/PPG-10/1 | 15.90 |
| | Dimethicone Polyglyceryl-2-isostearate (and) Hexyl Laurate | |
| | DC 2502 Cosmetic fluid: Cetyl Dimethicone | 1.90 |
| | Synthetic beeswax | 0.65 |
| | Hydrogenated castor oil | 0.30 |
| | Hexyl Laurate | 2.70 |
| B | Cyclopentasiloxane | 10.00 |
| C | Demineralized water | 47.50 |
| | Sodium chloride | 0.60 |
| | Propylene Glycol | 6.00 |
| | Preservative | 0.30 |
| D | Submica M: Mica | 5.05 |
| E | Titanium dioxide treated with lecithin | 8.05 |
| F | Glycerol | 0.74 |
| | Covacryl SJ5: Acrylates Copolymer | 0.13 |
| | Demineralized water | 0.59 |
| | Pigment composition containing yellow iron oxide and the | 0.34 |
| | additive of formula (III′) as described in Example 2 | |
| | Experiment 7 | |
| | Pigment composition containing the red iron oxide and the | 0.12 |
| | additive of formula (III′) as described in Example 2 | |
| | Experiment 8 | |
| | Pigment composition containing black iron oxide and the | 0.08 |
| | additive of formula (III′) as described in Example 2 | |
| | Experiment 2 | |

The ingredients of phase A were mixed and heated to 80° C., and then cooled to 50° C. The cyclopentasiloxane was then introduced into the mixture. The water, the salt, the propylene glycol and the preservative were mixed and heated to 50° C. and then slowly introduced into the oily phase with stirring (vortex). The mica and the titanium dioxide treated with lecithin were then introduced into the emulsion. The mixture of the three pigment compositions containing the additive (V′) was added into the mixture of water, glycerol and next Covacryl SJ5 was added. This phase was then introduced into the emulsion. The foundation has an off-white aspect and the beige color develops during application on the skin.

This example shows that the pigment composition containing sodium glycerophosphate (additive of formula (III′)) may be encapsulated and used in cosmetic compositions of the color-changing type.

### Example 11

### Shampoo Comprising a Pigment Composition Comprising the Additive of Formula (V′)

TABLE 17
Composition of the shampoo

| Ingredients | Mass (g) |
|---|---|
| Sodium Laureth Sulfate (28%) | 30.00 |
| Cocamidopropyl Betaine (30%) | 12.00 |
| Cocamide MIPA (and) Glyceryl Disterate | 3.00 |
| Polyquaternium-10 | 0.80 |
| Preservative | 0.30 |
| Tetrasodium EDTA | 0.20 |
| 20% citric acid in water | Adjust to pH = 6 |
| Demineralized water | Adjust to 100 |
| Pigment composition containing the red iron oxide and | 0.05 |
| the additive of formula (V′) as described in Example 4 | |
| Experiment 9 | |

Sodium Laureth Sulfate, Cocamidopropyl Betaine, water and the preservative were mixed and heated to 65° C. The mixture of Cocamide MIPA (and) Glyceryl Stearate was melted and then introduced into the previous mixture. Tetrasodium EDTA and then Polyquaternium-10 were introduced into the mixture. At room temperature, the pH was adjusted with citric acid.

This example illustrates the capability of the pigment composition containing phytic acid (additive of formula (V′)) of forming colored foams when it is introduced into a cosmetic composition of the shampoo type.

### Example 12

### Aqueous Correcting Foundation Comprising a Pigment Composition Comprising the Additive of Formula (V′)

TABLE 18
Composition of the aqueous correcting foundation

| | | Mass |
|---|---|---|
| | Ingredients | (g) |
| A | Pigment composition containing titanium dioxide and the | 23.6 |
| | additive of formula (V′) as described in Example 4 | |
| | Experiment 10 | |
| | Pigment composition containing black iron oxide and the | 0.7 |
| | additive of formula (V′) as described in Example 4 | |
| | Experiment 1 | |
| | Pigment composition containing red iron oxide and the | 1.9 |
| | additive of formula (V′) as described in Example 4 | |
| | Experiment 9 | |
| | Pigment composition containing yellow iron oxide and the | 3.80 |
| | additive of formula (V′) as described in Example 4 | |
| | Experiment 8 | |
| B | Demineralized water | 57.4 |
| | Preservative | 0.30 |
| C | Xanthan Gum (and) Hectorite (and) Cellulose | 0.30 |
| D | Saccaluronate LC: 1% hyaluronic acid in water | 5.00 |
| E | Serica 5 Mica | 2.00 |
| | Covabead PMMA2MUSI: Methyl Methacrylate Crosspolymer | 5.00 |
| | (and) silica | |

The preservative was introduced into the demineralized water, and then the four pigment compositions containing the additive (V′) were incorporated without grinding into the aqueous phase. The thickener based on xanthan gum was introduced with stirring, followed by Saccaluronate LC, Serica 5 and Covabead PMMA2MUSI.

### Example 13

### Cashmere Foundation Comprising a Pigment Composition Comprising the Additive of Formula (III′)

TABLE 19
Composition of the Cashmere foundation

| | | Mass |
|---|---|---|
| | Ingredients | (g) |
| A | Isononyl isononanoate | 10.00 |
| | Pigment composition containing black iron oxide and the | 0.25 |
| | additive of formula (III′) as described in Example 2 | |
| | Experiment 2 | |
| | Pigment composition containing red iron oxide and the | 0.45 |
| | additive of formula (III′) as described in Example 2 | |
| | Experiment 8 | |
| | Pigment composition containing yellow iron oxide and the | 1.88 |
| | additive of formula (III′) as described in Example 2 | |
| | Experiment 7 | |
| | Pigment composition containing titanium dioxide and the | 7.42 |
| | additive of formula (III′) as described in Example 2 | |
| | Experiment 10 | |
| B | Covalip 25: Candelila cera (and) Isopropyl palmitate (and) | 2.00 |
| | Ozokerite, Cetearyl ethylhexanoate (and) Isostearyl alcohol | |
| | (and) Cera carnauba (and) Myristyl lactate (and) Synthetic | |
| | beeswax (and) BHT | |
| | Macamat wax: Macadamia ternifolia seed oil (and) Ozokerite | 10.00 |
| | (and) Hydroxystearic acid (and) Stearic acid (and) Palmitic | |
| | acid | |
| | Covagloss: Polyethylene (and) Polybutene (and) Paraffinum | 8.00 |
| | liquidum | |
| | Hexyl laurate | 10.00 |
| C | Bentone 38: Disteardimonium hectorite | 4.00 |
| D | Cyclopentasiloxane | 10.00 |
| E | Isododecane | 26.50 |
| F | Covabead LH85: Methyl methacrylate crosspolymer | 3.00 |
| | Covafluid FS: Sodium stearyl fumarate | 1.00 |
| | Covamat: Mica (and) talcum (and) titanium dioxide (and) | 5.00 |
| | lauroyl lysine | |
| G | Propylene carbonate | 0.50 |

The four pigment compositions were dispersed in isononyl isononanoate with stirring. Phase B was heated to 90° C. until the waxes had melted and then cooled to 80° C. Phase A was incorporated into phase B with stirring until a homogeneous mixture was obtained. Phase C was introduced into the mixture kept at 80° C. Once the mixture had cooled to 60° C., phases D, E, F and G were introduced one after the other with stirring.

Examples 12 and 13 illustrate the capability of the pigment composition containing the additive of formula (III′) or (V′) of absorbing a large amount of oil in a cosmetic composition of the aqueous gel correcting foundation type for oily skins (Example 12) and an oily gel foundation (Example 13).

### Example 14

### Improvement of the Water Retaining Capacity of a Pigment Composition Comprising Red Iron Oxide as Compared with Red Iron Oxide

A 40% dispersion in water of the pigment composition of Example 2 reference 8 (comprising red iron oxide and the additive of formula (III′)) on the one hand, and a 40% dispersion in water of red iron oxide on the other hand were brought from an initial temperature of 25° C. to a temperature of 50° C. by heating at 1° C./min (a temperature of 50° C. reached in 25 mins), and the temperature of 50° C. was then maintained. The mass loss over time was tracked with a Perkin Elmer TGA7 thermogravimeter.

The appended figure shows that the 40% dispersion in water of the pigment composition according to the invention loses less rapidly the 40% water than the iron oxide dispersion.

### Example 15

### Dispersion of the Pigment Composition of Example 2/Experiment 2 in a Cosmetic Composition Comprising a Sequestering Agent and an Anionic Polymer

TABLE 20
Composition of the cosmetic composition

| | | Proportion |
|---|---|---|
| Phase | Component | (%) |
| A | Thickagent LC: Xanthan Gum (and) Hectorite (and) | 0.41 |
| | Cellulose | |
| B | Distilled water | 98.5 |
| | Trisodium citrate | 0.09 |
| C | Pigment composition containing black iron oxide and | 1.0 |
| | the additive of formula (III′) as described in Example 2 | |
| | Experiment 2 | |

The following operating procedure was followed:

Phase A was incorporated into phase B by stirring with a propeller at room temperature (25° C.). The stirring was maintained until a homogeneous gel was obtained. Phase C was incorporated by stirring with a propeller at room temperature. Stirring was maintained for 5 mins at 800 rpm.

Visual observation shows that the dispersion of the pigment composition into the obtained composition is excellent. The coloration of the composition is intense and homogeneous. The experiment was reproduced in the absence of sodium citrate (sequestering agent) on the one hand, by replacing the pigment composition of Experiment No. 2 of Example No. 2 with black iron oxide on the other hand.

Visual observation shows that the iron oxide is not as well distributed in the obtained compositions, and that their colorations are low.

### Example 16

### Foundation Comprising a Pigment Composition Comprising Iron Oxide, a Sequestering Agent and an Anionic Polymer

TABLE 20
Composition of the foundation

| | | % |
|---|---|---|
| PHASE | Ingredients | W/W |
| A | Preservative | 0.20 |
| | Sodium citrate | 0.10 |
| | Glycerin | 1.20 |
| | Aqua | 50.90 |
| B | Pigment composition containing black iron oxide and the | 7.85 |
| | additive of formula (III′) as described in Example 2 | |
| | Experiment 10 | |
| | Pigment composition containing yellow iron oxide and | 1.25 |
| | the additive of formula (III′) as described in Example 2 | |
| | Experiment 7 | |
| | Pigment composition containing black iron oxide and the | 0.25 |
| | additive of formula (III′) as described in Example 2 | |
| | Experiment 2 | |
| | Pigment composition containing red iron oxide and the | 0.65 |
| | additive of formula (III′) as described in Example 2 | |
| | Experiment 8 | |
| C | Natpure SF (Sucrose stearate, sucrose distearate) | 3.00 |
| | (Sensient) | |
| D | Covathick 2009 (microcrystalline cellulose, cellulose) | 18.40 |
| | (4% in water) (Sensient) | |
| E | Macadamia oil | 13.20 |
| F | Submica FL (Sensient) | 3.00 |

Phase B was dispersed into phase A with strong stirring. Phase C was added to the obtained mixture. A Covathick 2009 gel was prepared by dispersing the powder in water with a Rayneri apparatus and then by stirring for 1 minute at 13,500 rpm with an Ultraturrax apparatus. Phase D was then added to the mixture of phases A, B and C. The obtained mixture was then heated to 60° C. in a water container. Phase E was then added with stirring, followed by phase F. The obtained mixture was homogenized for 5 minutes.

1. A pigment composition in the form of a powder comprising:
an inorganic pigment or a mineral filler,
an additive of the following formula (I): 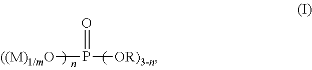
wherein:
n represents 1 or 2,
M represents H or a cation,
m represents 1 when M is H and m represents the valency of the cation when M is a cation,
R represents:
a group G selected from a saccharide or a group —[CH₂—CHR₁—O]_{q}—R₂ or —[CH₂—CH(CH₂OH)—O]_{q}—R₂ wherein:
q represents an integer from 1 to 1,000,
for each unit CH₂—CHR₁—O, R₁ represents independently H or a methyl,
R₂ represents H or an alkyl comprising from 1 to 3 carbon atoms, and,
a hydrocarbon chain comprising from 1 to 500 carbon atoms substituted with one or several G, phosphate (of formula OPO₃(M)_{2/m}) and/or hydroxyl (OH) groups.

2. The pigment composition according to claim 1, wherein the additive has the following formula (I′): 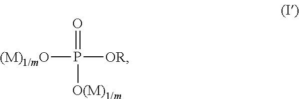
wherein M, m and R are as defined in claim 1.

3. The pigment composition according to claim 1, wherein the additive has one of the following formulae (II), (III) or (IV): 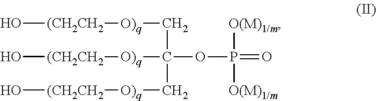
wherein M, m and q are as defined in claim 1, 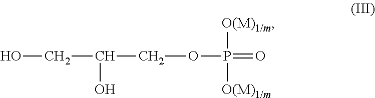
wherein M and m are as defined in claim 1, 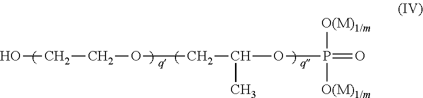
wherein M and m are as defined in claim 1 and q′ and q″ represent independently an integer from 0 to 1,000, such that the sum of q′ and q″ represent independently an integer from 1 to 1,000.

4. The pigment composition according to claim 1, wherein the additive has the following formula (V) or (VI): 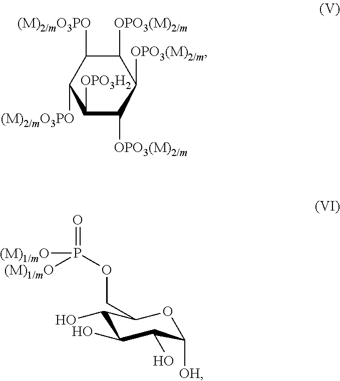
wherein M and n are as defined in claim 1.

5. The pigment composition according to claim 1, wherein the additive has one of the following formulae (II′), (III′), (IV′), (V′) or (VI′):

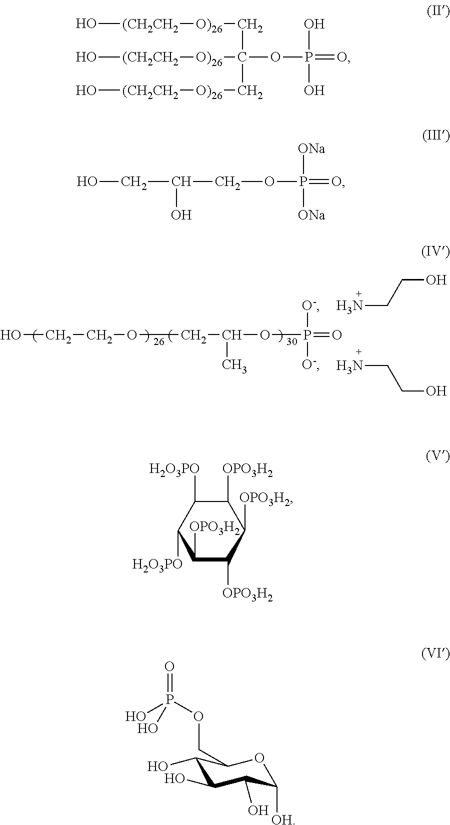

6. The pigment composition according to claim 1, wherein the pigment is selected from titanium dioxide, zinc dioxide, zirconium or cerium oxides, iron or chromium oxides, manganese violet, ultramarine blue, chromium hydrate and ferric blue, and mixtures thereof, preferably red iron oxide, yellow iron oxide, brown iron oxide, black iron oxide, titanium dioxide and mixtures thereof.

7. The pigment composition according to claim 6, wherein the pigment is an iron oxide, preferably red iron oxide, yellow iron oxide, brown iron oxide, black iron oxide and mixtures thereof.

8. The pigment composition according to claim 1, comprising from 0.01% to 20% by weight, typically from 0.5 to 10% by weight, preferably from 1 to 5% by weight, of an additive of formula (I) based on the weight of the combination pigment/additive of formula (I).

9. A method for preparing a pigment composition according claim 1, comprising the steps of:
a) mixing a pigment with an additive as defined above in a solvent,
b) optionally acidifying to a pH of less than or equal to 2 and/or addition of a salt after step a),
c) optionally basifying to a pH from 3 to 8 after step a) and the optional step b),
d) filtering the medium obtained after step a) and the optional steps b) and c), optionally followed by one or several steps of rinsing, drying and/or grinding for obtaining the pigment composition as a sold.

10. The cosmetic composition comprising a pigment composition according to claim 1.

11. The cosmetic composition according to claim 10, comprising a sequestering agent and a cosmetically acceptable anionic polymer, wherein the pigment is an iron oxide, preferably red iron oxide, yellow iron oxide, brown iron oxide, black iron oxide and mixtures thereof.

12. The cosmetic composition according to claim 11, wherein the sequestering agent is selected from phytic acid, citric acid, gluconic acid, pentetic acid, ethylene diaminetetraacetic acid (EDTA), cyclodextrin, salts thereof and a mixture thereof, preferably citric acid or one of its salts.

13. The cosmetic composition according to claim 11, wherein the cosmetically acceptable anionic polymer is xanthan gum, carboxymethylcellulose or a polyacrylate.
